(19) United States
(12) Patent Application Publication
(10) Pub. No.: US 2016/0354570 A1
(43) Pub. Date: Dec. 8, 2016

(54) Oxygen Cord Retractor and Organizer

(71) Applicant: Lorelyn Cajucom Arroyo, Saint Petersburg, FL (US)

(72) Inventor: Lorelyn Cajucom Arroyo, Saint Petersburg, FL (US)

(21) Appl. No.: 15/084,250

(22) Filed: Mar. 29, 2016

Related U.S. Application Data
(63) Continuation of application No. 62/172,591, filed on Jun. 8, 2015.

(51) Int. Cl. A61M 16/06; A61M 16/08; B65H 75/44; A61M 16/10

(52) U.S. Cl. CPC A61M 16/06; A61M 16/101; A61M 16/08; B65H 75/4471; A61M 2209/088

(57) ABSTRACT

An oxygen cord retractor and organizing apparatus is described. The apparatus is equipped with a housing, swivel base, a string or rope, spring-based retraction system, and clips. The oxygen cord retractor is configured to keep oxygen cord, hose, or tubing in a safely organized and coiled state both when stored and during use. The string or rope is disposed to extend within the coiled oxygen hose, and is configured to extend and retract via the spring-based retraction system housed within the housing of the apparatus. An oxygen cord organizer is disposed atop the housing, which facilitates the organization of the oxygen cord when not in use, and functions as a mounting point for the oxygen cord to the oxygen concentrator or oxygen cylinder/tank. The string is configured to extend and retract with ease from the housing via the spring-based retraction system, and remains taught during use.

This application is a non-provisional application of provisional patent application No. 62/172,591, filed on Jun. 8, 2015, and priority is claimed thereto.

### FIELD OF THE PRESENT INVENTION

The present invention relates generally to oxygen dispensing equipment, and more specifically relates to an apparatus configured to facilitate the retraction, organization, and storage of an oxygen cord, hose, or tubing that accompanies conventional oxygen breathing systems.

### BACKGROUND OF THE PRESENT INVENTION

Often, when people wear oxygen, it is commonly a pain. The oxygen is placed on a wheeled cart, similar to an IV, and must be brought with the user wherever he or she travels. The cord can often get tangled or stuck on household objects, and must be taken with the user outside of the home for proper consistent use, or to be available in emergencies. Use of the oxygen system is cumbersome, and is belabored by poorly fashioned cylinder carts that can be toppled as the user moves about a room.

As the safety and security of one's oxygen tube and system is important for the health and wellbeing of the user, it is critical to maintain, use, and store the oxygen tube such that it is free from damage, debris, kinks, or obstruction. Often, when storing an oxygen system, the user must manually coil the non-rigid oxygen tube for organizing the system, which can be laborious.

Thus, there is a need for an oxygen cord retractor configured to keep the oxygen cord taught, organized, untangled, and readily accessible during use and when stored with minimal effort. Such a device is preferably configured to maintain the oxygen tube or cord in a coiled, taught, and untangled state, helping to prevent tangles, snares, and damage to the oxygen tube or cord during use.

### SUMMARY OF THE PRESENT INVENTION

The present invention is an oxygen cord retraction, organizing, and storage apparatus configured to ensure safe use and prolonging the life of the oxygen cord when used in tandem with an oxygen dispensing system, such as a tank, cylinder, or other compressor.

The present invention provides novel features to enhance the user experience of those that require oxygen. For example, the present invention is equipped with a swivel base configured to enable the anchor point of the oxygen tube to consistently face the user as he or she moves about a room. The present invention is equipped with a housing disposed atop the base, which is configured to organize and store coiled oxygen tube when not in use. An anchor string is connected to a spring-based tensioner disposed within the housing of the present invention, and is configured to retract when not in use. The spring-based tensioner keeps the anchor string taught, which is affixed to the administration end (exhaust end) of the oxygen tube, near the user, preferably via a strap and/or clip. The anchor string is disposed such that it runs through the center of the coil of the coiled oxygen tube, and extends from the housing of the present invention. As the user walks towards the oxygen concentrator or tank, which is disposed next to the present invention, the anchor string is retracted into the housing via the spring-based tensioner, helping to keep the oxygen tube organized in a manageable coil. As the user walks away from the housing of the present invention, the anchor string is released from the housing via an anchor string outlet. Due to the swivel base of the present invention, the lengthening of the anchor string of the oxygen cord retractor device, and the uncoiling of the oxygen tube around the anchor string is facilitated. The user can walk in any direction, within 360 degrees around the present invention, without leaving a messy trace of the oxygen tubing in the user's path, helping to eliminate the risk of stepping on the oxygen tube and/or tripping during walking.

### BRIEF DESCRIPTION OF THE DRAWINGS

The present invention will be better understood with reference to the appended drawing sheets, wherein:

FIG. 1 is the top front view of a primary embodiment of the hose organizer and coiling tube of the present invention.

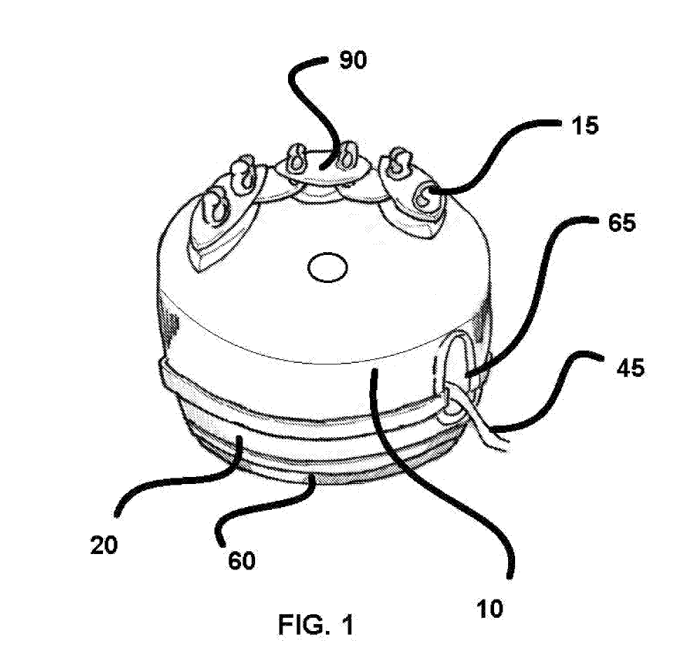

FIG. 2 depicts a view of the top of the preferred embodiment of the oxygen cord retractor of the present invention.

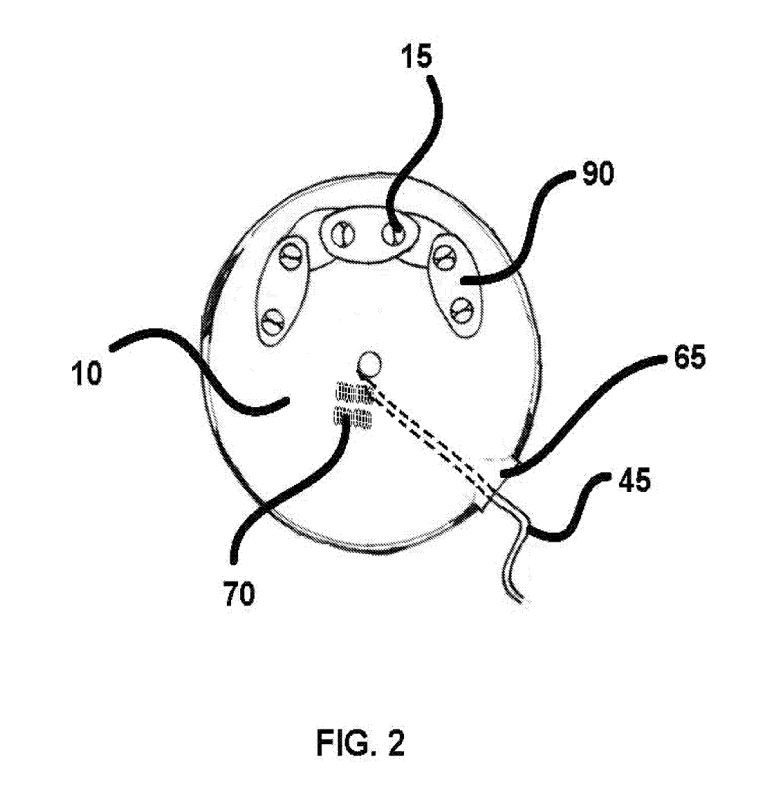

FIG. 3 displays a front layout view of the oxygen cord retractor of the present invention in use.

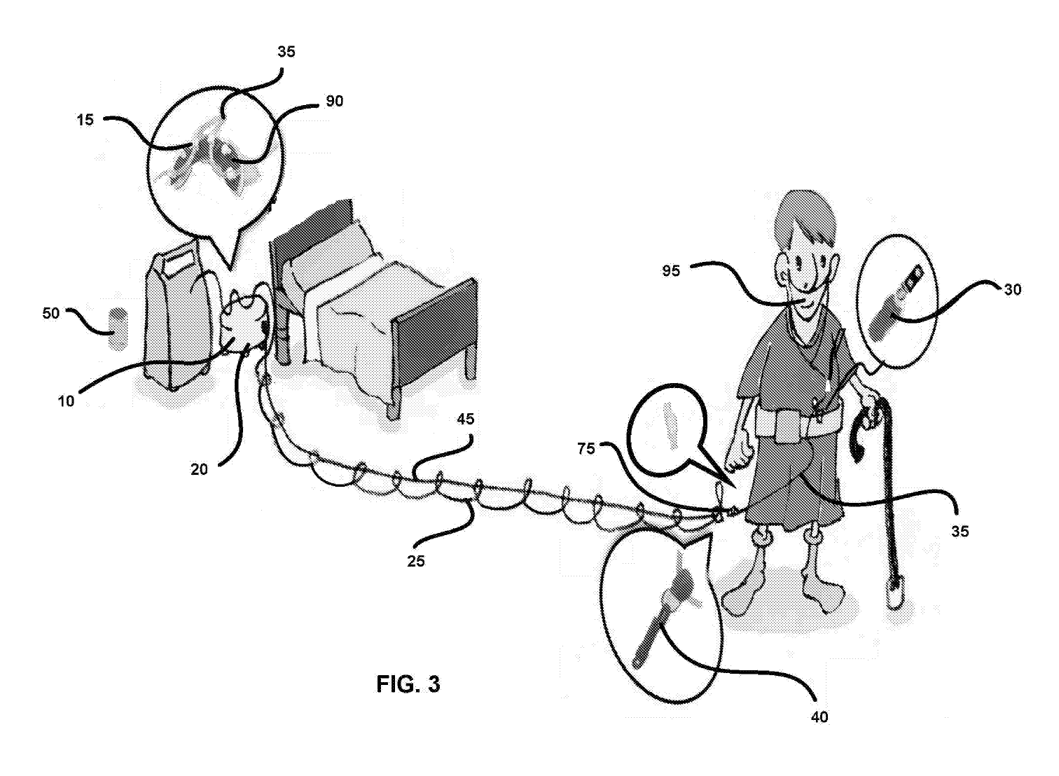

FIG. 4 exhibits a flow chart detailing the process of use of the present invention.

### DETAILED DESCRIPTION OF THE PREFERRED EMBODIMENT

The present invention is an oxygen tube retention, retraction, storage, and organization system for personal use by a user. The preferred embodiment of the present invention is equipped with a retractor housing (10), a base (20), coiled oxygen tube channel (25), an oxygen tube (35), a clothing-to-tube clip (30), a string-tube clip (40), an anchor string (45), an anchor string outlet (65), a coiling tube (50), a swivel mechanism (60), and a spring-based tensioner (70). The spring of the spring-based tensioner (70) is preferably a constant-force spring, and is disposed within the retractor housing (10). The base (20) is disposed below the retractor housing (10), and is equipped with the swivel mechanism (60) to facilitate a pivot of the coiled oxygen tube channel (25) of up to 360 degrees. A tether affixed below the base (20) of the present invention is preferably employed to connect the present invention to an oxygen tank, compressor, concentrator, or similar oxygen administration/storage device. The tether prevents the oxygen compressor from tipping over in the event that the user walks far enough away from the present invention to cause the anchor string (45) to become fully extended from the retractor housing (10).

At least one wheel is preferably disposed beneath the base (20) of the present invention to provide for easy relocation of the present invention. Additionally, the present invention may be equipped with a rubber coating or rubber feet under the base (20) to ensure the present invention maintains adequate traction with the floor on which it preferably rests. Alternate embodiments of the present invention may employ at least one suction cup to help ensure that the present invention maintains in the preferred position. The retractor housing (10) is preferably equipped with a handle disposed on the rear to facilitate the transport of the present invention for relocation.

In the primary embodiment of the present invention, the oxygen tube (35) within the coiled oxygen tube channel (25) is affixed directly to an oxygen tank, and is preferably woven through at least one tube mount (15) to secure the oxygen tube channel (25) containing the oxygen tube (35) to the retractor housing (10). In one alternate embodiment of the present invention, the oxygen tube (35) is in communication with the base (20) of the present invention via a first valve disposed atop the retractor housing (10). A second valve is disposed below the swivel mechanism (60) of the base (20), and facilitates the connection of the present invention to the oxygen tank, oxygen cylinder, or oxygen concentrator. An oxygen tube organizer (90) is disposed atop the retractor housing (10), and acts to secure the coiled oxygen tube (25) firmly to the retractor housing (10) via the at least one tube mount (15). In the preferred embodiment of the present invention, the oxygen tube (25) is connected directly to the oxygen cylinder and to the oxygen mask, and need not be attached via the first valve and the second valve to the base (20) for the organization and retraction features of the present invention to function. The coiling tube (50) is preferably used to manually coil the oxygen tube (25) for storage when not in use. The coiling tube (50) may preferably be placed on top of the retractor housing (10).

The anchor string (45) of the present invention ensures that the coiled oxygen tube (25) remains taught as the user moves about a room, as the anchor string (45) itself is held taught, retracted via the spring-based tensioner (70). The anchor string (45) is in communication with the spring-based tensioner (70) of the present invention, and preferably exits the retractor housing (10) via the anchor string outlet (65). A quick release clip is preferably used to secure the anchor string (45) to an administering end (75) of the coiled oxygen tube channel (25). Alternate embodiments of the present invention may be equipped with an adjustable click belt configured to be worn around the waist of the user to facilitate the connection of the oxygen tube (35) to the user via the clothing-to-tube clip (30). Near this juncture, the coiled oxygen tube channel (25) and/or oxygen tube (35) is connected to the clothing of the user via the clothing-to-tube clip (30). It should be understood that the coiled oxygen tube channel (25) is preferably rigid such that it retains a coiled shape when elongated and stretched out.

The length and diameter of the anchor string (45) of the present invention may vary, however it is envisioned that the anchor string (45) is at least 25 feet long, and has a diameter larger than a quarter inch. The user may need to manually feed the oxygen tube (35) into the coiled oxygen tube channel (25) if it is not preconfigured. The administering end (75), otherwise known as the exhaust end, is connected to an oxygen mask (95) (or oxygen cannula) to dispense the oxygen to the user. The oxygen mask (95) is preferably conventional.

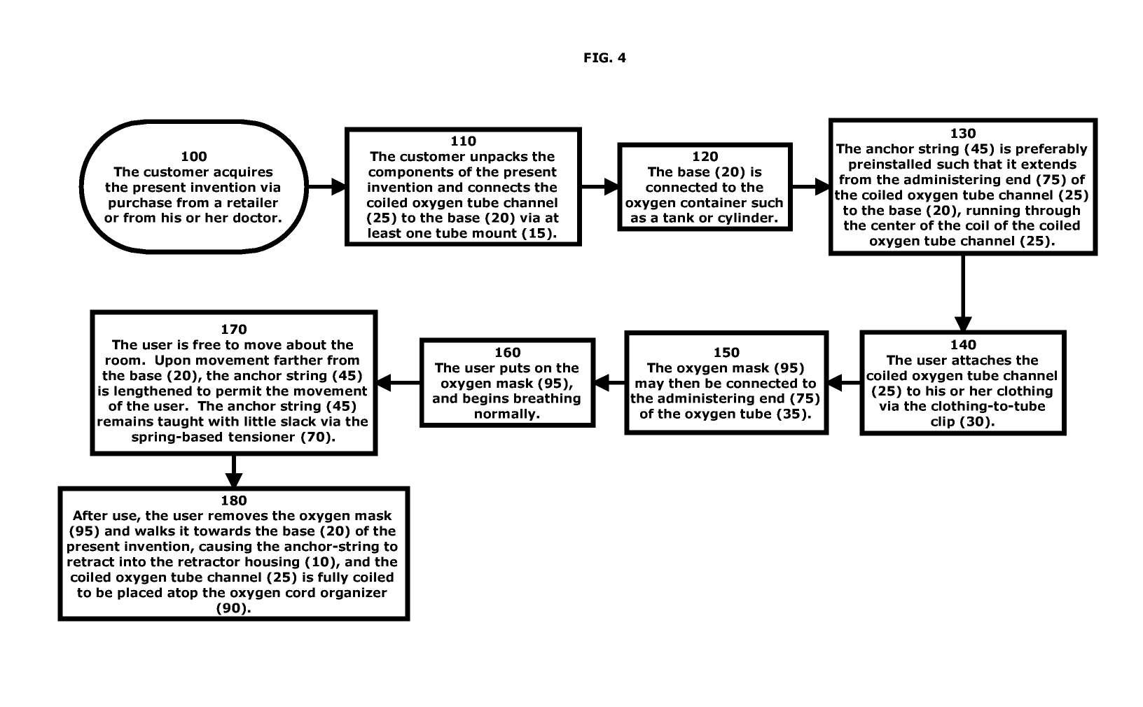

The process of use of the present invention, as depicted in FIG. 4, is preferably as follows:
1. The customer acquires the present invention via purchase from a retailer or from his or her doctor. (100)
2. The customer unpacks the components of the present invention and connects the coiled oxygen tube channel (25) to the base (20) via at least one tube mount (15). (110)
3. The base (20) is connected to the oxygen container such as a tank or cylinder. (120)
4. The anchor string (45) is preferably preinstalled such that it extends from the administering end (75) of the coiled oxygen tube channel (25) to the base (20), running through the center of the coil of the coiled oxygen tube channel (25). (130)
5. The user attaches the coiled oxygen tube channel (25) to his or her clothing via the clothing-to-tube clip (30). (140)
6. The oxygen mask (95) may then be connected to the administering end (75) of the oxygen tube (35). (150)
7. The user puts on the oxygen mask (95) (or oxygen cannula), and begins breathing normally. (160)
8. The user is free to move about the room. Upon movement farther from the base (20), the anchor string (45) is lengthened to permit the additional movement of the user. The anchor string (45) remains taught with little slack via the spring-based tensioner (70). (170)
9. After use, the user removes the oxygen mask (95) (or oxygen cannula) and walks it towards the base (20) of the present invention, causing the anchor-string to retract into the retractor housing (10), and the coiled oxygen tube channel (25) is fully coiled to be placed atop the oxygen cord organizer (90) in and/or around the coiling tube (50). (180)

Alternate embodiments of the present invention include variations on the size and shape of the anchor string (45) employed. For example, a strap, cable, band, or similar retractable and coiled item could be used in lieu of a string. Additionally, some embodiments of the base (20) of the present invention may be equipped with wheels which may be locked in place. Wheels at the base (20) of the present invention facilitate transport of the present invention between rooms if needed.

It should be understood that there may be times during which the coils of the coiled oxygen tube (25) will vary in size and will be uneven. In this event, the patient must move the anchor string (45) along with the coiled oxygen tube (25) up, down, and sideways to promote an even coiling of the coiled oxygen tube (25). It should be noted that the present invention is preferably sold with optional components that may be purchased separately. It is envisioned that the coiling tube (50), oxygen mask (90), coiled oxygen tube channel (25) and oxygen tube (35) may be purchased separately from the oxygen cord retractor, however they are required for the proper functioning of the present invention.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. An oxygen cord retractor device for an oxygen tube comprising:
a housing;
a base, said housing disposed above said base;
a swivel mechanism, said swivel mechanism disposed between said housing and said base;
wherein said housing is configured to pivot about said swivel mechanism;
a coiled oxygen tube channel, said coiled oxygen tube channel configured to receive the oxygen tube;
a spring-based tensioner, said spring-based tensioner disposed within said housing;
wherein said spring-based tensioner employs a constant force spring;
an anchor string, said anchor string in communication with said spring-based tensioner;
an anchor string outlet, said anchor string outlet disposed on said housing;
wherein said anchor string is configured to extend from said housing via said anchor string outlet within the center of the coil of said coiled oxygen tube channel containing the oxygen tube;
an oxygen mask, said oxygen mask in communication with the oxygen tube; and
an oxygen cord organizer, said oxygen cord organizer disposed atop said housing.

2. The device of claim 1, further comprising:
a clothing-to-tube clip, said clothing-to-tube clip in direct communication with said coiled oxygen tube channel;
wherein the oxygen tube is equipped with input end and an exhaust end;
wherein said input end is connected to an oxygen dispenser; and
wherein said exhaust end is connected to said oxygen mask.

3. The device of claim 1, further comprising:
at least one tube mount;
wherein said at least one tube mount is disposed on said oxygen cord organizer;
wherein said at least one tube mount is configured to secure the oxygen tube to said housing; and
wherein said oxygen cord organizer buffers tension between said oxygen mask and an oxygen dispenser.

4. The device of claim 1, further comprising:
a string-tube clip, said string-tube clip in communication with said anchor string and said coiled oxygen tube channel; and
wherein said string-tube clip loosely binds said anchor string to the oxygen tube.

5. The device of claim 3, wherein said oxygen dispenser is one of the following:
an oxygen tank, an oxygen compressor, or an oxygen concentrator.

6. The device of claim 2, further comprising:
at least one tube mount;
wherein said at least one tube mount is disposed on said oxygen cord organizer;
wherein said at least one tube mount is configured to secure the oxygen tube to said housing; and
wherein said oxygen cord organizer buffers tension between said oxygen mask and an oxygen dispenser.

7. The device of claim 2, further comprising:
a string-tube clip, said string-tube clip in communication with said anchor string and said coiled oxygen tube channel; and
wherein said string-tube clip loosely binds said anchor string to the oxygen tube.

8. An oxygen tube retractor device comprising:
a housing;
a base, said housing disposed above said base;
a swivel mechanism, said swivel mechanism disposed between said housing and said base;
wherein said housing is configured to pivot about said swivel mechanism;
a coiled oxygen tube channel, said coiled oxygen tube channel configured to receive the oxygen tube;
a spring-based tensioner, said spring-based tensioner disposed within said housing;
wherein said spring-based tensioner employs a constant force spring;
an anchor string, said anchor string in communication with said spring-based tensioner;
an anchor string outlet, said anchor string outlet disposed on said housing;
wherein said anchor string is configured to extend from said housing via said anchor string outlet within the center of the coil of said coiled oxygen tube channel containing the oxygen tube;
an oxygen mask, said oxygen mask in communication with the oxygen tube;
an oxygen cord organizer, said oxygen cord organizer disposed atop said housing;
a clothing-to-tube clip, said clothing-to-tube clip in direct communication with said coiled oxygen tube channel;
wherein the oxygen tube is equipped with input end and an exhaust end;
wherein said input end is connected to an oxygen dispenser;
wherein said exhaust end is connected to said oxygen mask;
at least one tube mount;
wherein said at least one tube mount is disposed on said oxygen cord organizer;
wherein said at least one tube mount is configured to secure the oxygen tube to said housing;
wherein said oxygen cord organizer buffers tension between said oxygen mask and said oxygen dispenser;
a string-tube clip, said string-tube clip in communication with said anchor string and said coiled oxygen tube channel; and
wherein said string-tube clip loosely binds said anchor string to the oxygen tube.
